(19) United States
(12) Patent Application Publication
(10) Pub. No.: US 2026/0069583 A1
(43) Pub. Date: Mar. 12, 2026

(54) COMPOSITION FOR THE TREATMENT OF ACNE

(71) Applicant: MEDSPA S.R.L., MILANO (IT)

(72) Inventor: Camilla D'ANTONIO, MILANO (IT)

(21) Appl. No.: 19/108,851

(22) PCT Filed: Sep. 4, 2023
(86) PCT No.: PCT/IB2023/058723

§ 371 Date: Mar. 5, 2025

(30) Foreign Application Priority Data
Sep. 5, 2022 (IT) 102022000018138

(51) Int. Cl. A61K 31/455; A61K 8/19; A61K 8/34; A61K 8/368; A61K 8/44; A61K 8/67; A61K 9/00; A61K 31/05; A61K 31/198; A61K 31/60; A61K 33/38; A61P 17/10; A61Q 19/00

(52) U.S. Cl. CPC A61K 31/455; A61K 8/19; A61K 8/347; A61K 8/368; A61K 8/442; A61K 8/675; A61K 9/0014; A61K 31/05; A61K 31/198; A61K 31/60; A61K 33/38; A61P 17/10; A61Q 19/008

(57) ABSTRACT

It is the object of the present invention a composition for the treatment of acne.

The present invention applies to the treatment of acne.

Acne is a condition of inflammation of the pilosebaceous follicles.

In addition to representing a medical condition, it often also is an aesthetic problem, due to the presence of comedones and pustules, often commonly referred to as pimples, on the face and other parts of the body, such as the back, chest, and neck.

There can be different causes that determine acne. While in adolescence it can be due to hormonal activity, stress can cause acne to arise even in adulthood.

Although hygiene, environmental conditions, such as pollution, and poor nutrition are circumstances which can determine the appearance or aggravate the condition thereof, certainly the most relevant component is familiarity.

Therefore, the treatment of acne requires changing the lifestyle and using products specifically adapted for this purpose.

For example, facial cleansing products must be particularly gentle so as not to increase the state of inflammation, but must also be capable of exerting a sufficiently effective cleansing action in freeing the follicles.

### SUMMARY OF THE INVENTION

The present inventors have surprisingly found that a specific qualitative combination of active ingredients is capable of providing surprising results in the treatment of acne.

### OBJECT OF THE INVENTION

In a first object, the present invention describes a topical anti-acne composition.

In a second object, the present invention describes a topical formulation comprising the composition of the invention.

In a preferred aspect, such a formulation is a cream.

In a third object, the present invention describes the topical composition for the medical use in the prevention and/or treatment of acne.

In an aspect of the invention, such a use is in association with one or more other topical and/or oral compositions.

In another object, the present invention describes the cosmetic use of the topical composition of the invention for the prevention and/or treatment of imperfections caused by acne.

### DETAILED DESCRIPTION OF THE INVENTION

In accordance with a first object of the present invention, a topical anti-acne composition is described.

In particular, the composition of the invention comprises:
niacinamide,
azeloglycine,
salicylic acid,
Bakuchiol, and
silver.

For the purposes of the present invention, niacinamide is present in a concentration of about 1-7% (w/v).

In a preferred aspect of the invention, niacinamide is present in a concentration of about 5% (w/v).

As for azeloglycine (monopotassium azelaoyldiglycinate, INCI potassium azeloyl diglycinate), this is represented by the compound of formula:

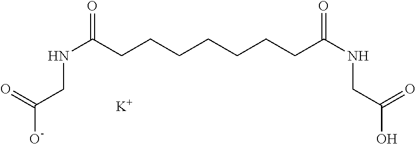 [SMILES: O=C(O)CNC(=O)CCCCCCCC(=O)NCC(=O)O ; [K+]]

For the purposes of the present invention, it is present in a concentration of about 1-5% (w/v).

In a preferred aspect of the invention, azeloglycine is present in a concentration of about 3% (w/v).

For the purposes of the present invention, salicylic acid is present in a concentration of about 0.3-0.9% (w/v).

In a preferred aspect of the invention, salicylic acid is present in a concentration of about 0.5% (w/v). As for bakuchiol, this is represented by the compound phenol 4-[1E, 3S)-3-ethenyl-3,7-dimethyl-1,6-octadienyl (also marketed under the name Sytenol®A) of formula:

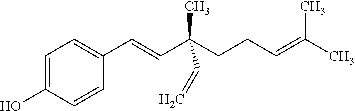 [SMILES: C=C[C@@](C)(/C=C/c1ccc(O)cc1)CCC=C(C)C]

For the purposes of the present invention, bakuchiol is present in a concentration of about 0.1-1% (w/v).

In a preferred aspect of the invention, bakuchiol is present in a concentration of about 0.4% (w/v).

As for silver, this is in powder form.

According to a preferred aspect, silver is in the form of a microporous powder, where an example of such a form is represented by the product MICROSILVER BGTM (Bio-Gate AG).

For the purposes of the present invention, silver is present in a concentration of about 0.01%-0.1% (w/v).

According to an aspect of the present invention, the composition comprises niacinamide, azeloglycine, salicylic acid, Bakuchiol and silver as the only active ingredients.

In a second object, the present invention describes a topical formulation comprising the composition of the invention.

Therefore, in this regard the formulation comprises, together with the composition described, acceptable excipients which are suitable for a topical formulation.

Those skilled in the art are able to prepare such a formulation based on their technical knowledge in the field; stabilizers, emulsifiers, antimicrobials, dyes and fragrances can thus be included, as deemed necessary.

Possible formulations comprise a cream, a solution, an emulsion, a lotion, or a gel.

In a preferred aspect, the formulation of the invention is represented by a cream.

In accordance with a third object, the present invention describes the above topical composition for the medical use in the prevention and/or treatment of acne.

For the aspects of the present invention, the subject suffering from acne is a young, adolescent or adult male or female subject and without race limitations.

For the aspects of the present invention there is no limitation to the type of cause of the prevented or treated which can be due to hormonal causes, stress, pharmacological care, sun exposure, etc.

According to an aspect of the invention, the medical use of the composition of the invention is described in association with one or more further topical formulations.

Such topical formulations can be represented by products having cleansing and/or sebum regulating and/or astringent activity.

Such formulations can be employed prior to applying the formulation of the composition of the invention.

According to a further aspect of the invention, such a medical use can be in further association with oral compositions.

In accordance with an aspect of the invention, the above topical composition is described as an adjuvant in the medical use for the prevention and/or treatment of acne.

In accordance with a third object, the present invention describes the cosmetic use of the above composition for the prevention and/or treatment of skin imperfections caused by acne.

In particular, said cosmetic use for the prevention and/or treatment of skin imperfections is to obtain one or more of the following effects:
reduction of pimple size,
reduction in the number of pimples,
reduction of erythema,
reduction of sebum production.

According to an aspect of the invention, the cosmetic use of the composition of the invention is described in association with one or more further topical formulations.

Such topical formulations can be represented by products having cleansing and/or sebum regulating and/or astringent activity.

Such formulations can be employed prior to applying the formulation of the composition of the invention.

According to a further aspect of the invention, such a cosmetic use can be in further association with oral compositions.

A cream formulation comprising the composition of the invention was tested in instrumental and clinical tests in order to evaluate the effects thereof, and the results obtained are detailed below.

### Instrumental Tests

14 volunteers aged 19-39 years having an acne-prone skin with imperfections applied the cream of the invention twice daily for 60 days.

Device: Visiopor® PP 34, Mexameter MX 18, Sebumeter, Vectra H2.

The results obtained are shown in the following table.

| | | Number of | | |
|---|---|---|---|---|
| | Pimple size | pimples | erythema | sebum |
| Improvement | 17 | 27 | 10 | 22 |
| after 30 days | | | | |
| (%) | | | | |
| Improvement | 20 | 29 | 15 | 36 |
| after 60 days | | | | |
| (%) | | | | |

### Dermatological Clinical Evaluation.

14 volunteers aged 19-39 years having an acne-prone skin with imperfections applied the cream of the invention twice daily for 60 days.

The results obtained are shown in the following table.

| | | | Pimple | Greasy |
|---|---|---|---|---|
| | | | density and | and oily |
| | Pimples* | Scars/spots** | distribution | skin |
| Improvement | 71 | 57 | 57 | 79 |
| after 60 days | | | | |
| (%) | | | | |

*amount of pre-inflammatory and non-inflammatory primary lesions
**amount of secondary lesions such as scars, pigmentation alterations, post-inflammatory erythema

Self-assessment.

14 volunteers aged 19-39 years having an acne-prone skin with imperfections applied the cream of the invention twice daily for 60 days.

The results obtained regarding the other reported effects are shown in the following table.

| | Number of subjects who |
|---|---|
| Effect | perceived the effect |
| overall skin improvement | 70 |
| texture improvement | 70 |
| soothing and purifying action | 70 |
| sebum-normalizing action | 100 |
| pleasant sensation on the skin during | 100 |
| application | |
| fragrance pleasantness and degree of | 86 |
| absorption | |

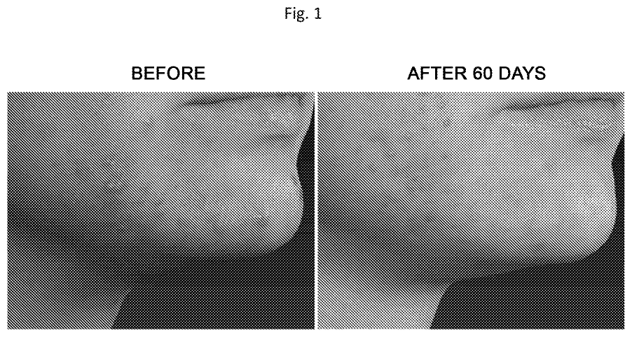

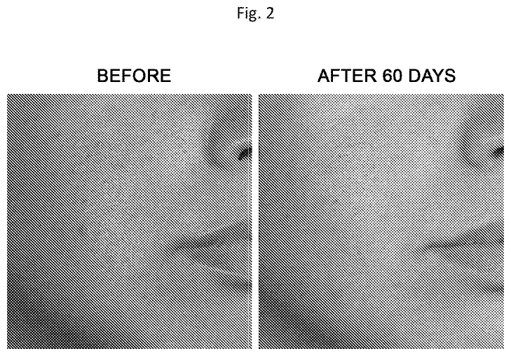

The images of FIGS. 1 and 2 show the condition of the skin on chin and cheeks before (left) and after 60 days (right) of treatment of the present invention. Images obtained with Vectra H2.

From the above description, the advantages offered by the present invention will be immediately apparent to those skilled in the art.

In particular, the composition of the invention surprisingly exhibited excellent and surprising properties in the treatment, also as an adjuvant, of acne, which properties go far beyond what could be expected from the combination of the active ingredients contained therein.

In addition to the ability to treat acne, the subjects who participated in the clinical trials highlighted and reported other positive effects, such as: overall skin improvement, texture improvement, soothing and purifying action, sebum-normalizing action, pleasant sensation on the skin during application and degree of absorption. Such effects were reported by at least 70% of subjects. The composition of the invention is stable and allows the addition of fragrances.

Moreover, the described composition is not photosensitizing.

1. A topical composition comprising niacinamide, azeloglycine, salicylic acid, Bakuchiol and silver.

2. A composition according to the claim 1, wherein niacinamide is present in a concentration of about 1-7% (w/v), and preferably of about 5% (w/v).

3. A composition according to claim 1, wherein azeloglycine is present in a concentration of about 1-5% (w/v), and preferably in a concentration of about 3% (w/v).

4. A composition according to claim 1, wherein salicylic acid is present in a concentration of about 0.3-0.9% (w/v), and preferably of about 0.5% (w/v).

5. A composition according to claim 1, wherein bakuchiol is present in a concentration of about 0.1-1% (w/v), and preferably of about 0.4% (w/v).

6. A composition according to claim 1, wherein silver is present in a concentration of about 0.01-0.1% (w/v).

7. A topical formulation comprising the composition according to claim 1 and dermatologically acceptable excipients.

8. A topical formulation according to claim 1, which is in the form of a cream, a solution, an emulsion, a lotion, or a gel.

9. (canceled)

10. (canceled)

11. A cosmetic method for the treatment and/or prevention of skin imperfections caused by acne comprising the use of the topical composition according to claim 1.

12. The cosmetic method according to the claim 11, wherein said use is to obtain one or more of the following effects:
reduction of the size of pimples,
reduction in the number of pimples,
reduction of erythema,
reduction of sebum production.

13. A method for the prevention and/or treatment of acne comprising the use of a topical composition according to claim 1.

14. A method for the prevention and/or treatment of acne comprising the use of a topical composition according to a topical formulation according to claim 7.

15. A method for the prevention and/or treatment of acne comprising the use of a topical composition according to claim 1, wherein said topical composition or said topical formulation are as an adjuvant in the prevention and/or treatment of acne.

16. A method for the prevention and/or treatment of acne comprising the use of a topical composition according to a topical formulation according to claim 7, wherein said topical composition or said topical formulation are as an adjuvant in the prevention and/or treatment of acne.

17. A cosmetic method for the treatment and/or prevention of skin imperfections caused by acne comprising the use of the topical composition according to the topical formulation according to claim 7.
